(19) United States
(12) Patent Application Publication
(10) Pub. No.: US 2023/0165601 A1
(43) Pub. Date: Jun. 1, 2023

(54) TONGUE DEBRIDING APPARATUS

(71) Applicants: Adrian Carrington, Granite Bay, CA (US); Marsha Henry, Granite Bay, CA (US)

(72) Inventors: Adrian Carrington, Granite Bay, CA (US); Marsha Henry, Granite Bay, CA (US)

(21) Appl. No.: 18/161,793

(22) Filed: Jan. 30, 2023

Related U.S. Application Data
(63) Continuation of application No. PCT/US2021/043720, filed on Jul. 29, 2021.
(60) Provisional application No. 63/059,236, filed on Jul. 31, 2020.

(51) Int. Cl. A61B 17/24; A46D 1/00; A46B 9/02; A46B 15/00

(52) U.S. Cl. CPC A61B 17/244; A46D 1/0207; A46B 9/02; A46B 15/0053; A46B 15/0051; A61B 2217/005; A61B 2017/320012

(57) ABSTRACT

Surface debriding systems and instruments are provided that efficiently and effectively dislodge and remove debris, coatings, microorganisms and fluids from the dorsum of the tongue or other surfaces. The instrument has a debriding surface with cleaning elements and a number of spaced apart openings to a hollow interior that is configured to couple with a vacuum source. The shape of the openings may be configured to amplify suction force from the vacuum source. The system also has a vacuum source and a collecting vessel with backflow valves to store removed material and to stop movement of material into the vacuum source or back out to the instrument. The system can be self-contained or the instrument may be coupled to a dental high-volume evacuator (HVE) vacuum source.

### CROSS-REFERENCE TO RELATED APPLICATIONS

This application claims priority to, and is a 35 U.S.C. § 111(a) continuation of, PCT international application number PCT/US2021/043720 filed on Jul. 29, 2021, incorporated herein by reference in its entirety, which claims priority to, and the benefit of, U.S. provisional patent application Ser. No. 63/059,236 filed on Jul. 31, 2020, incorporated herein by reference in its entirety. Priority is claimed to each of the foregoing applications.

The above-referenced PCT international application was published as PCT International Publication No. WO 2022/026722 A1 on Feb. 3, 2022, which publication is incorporated herein by reference in its entirety.

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to human or animal oral hygiene devices, systems and methods and more particularly to an apparatus and methods for safely and effectively dislodging and removing microorganisms and debris from the dorsum of the tongue and other surfaces.

### 2. Background

The upper surface (dorsum) of the tongue has many papillae, furrows and groves that can collect debris and plaque as well as harbor undesirable microorganisms that can generate halitosis and foul tastes in the mouth. In addition, the tongue can accumulate materials from sinus drainage that can also contribute to poor oral hygiene.

Visits to the dentist for the typical continuing care appointments entail scaling (scraping) of plaque and calculus from the tooth surface followed by polishing of any residual stain away from the tooth. This process does not completely eliminate bacteria from tooth surfaces, but it may reduce and disturb the microbiota load. Although it is well known that the tongue presents crevices between the papillae on the surface where dental procedure debris accumulates and can allow microbes to proliferate, this dental debris is usually ignored by the clinical team. Furthermore, tongue cleaning is often not common in the daily home hygiene regimen for most patients, leaving the dental debris intact. This represents a void in oral hygiene care that needs to be addressed.

Because the tongue is one of the largest reservoirs of microbiota in the oral cavity, various tongue cleaners have been developed to attempt to loosen and remove debris from the folds and grooves as well as other materials that may coat the tongue. Tongue cleaning devices in the art include a variety of washes, tongue scrapers, tongue brushes and other structures disposed on a toothbrush handle at the end opposite the tooth-brush bristles. However, brushes and scrapers may not adequately reach the bottom of the grooves and furrows allowing material to remain. Furthermore, the material dislodged by these devices may not be completely removed from the mouth and may be swallowed by the patient or allow repopulation of undesirable microorganisms. Such devices have also been known to stimulate the gag reflex resulting in an incomplete cleaning of the tongue because the procedure is not well tolerated by the patient or user.

Therefore, there is a need for a tool for debriding tongue debris and micro biota that is inexpensive, effective and easy for dentists and dental clinical staff to use.

### BRIEF SUMMARY

Devices, systems and methods are provided for effective, efficient and safe removal of accumulated debris and microorganisms from the irregular surfaces, folds and grooves of the dorsum of the tongue as well as other surfaces of the mouth. The preferred system has a debriding head that is connected to a vacuum source so that dislodged debris and fluids are removed from the mouth for disposal. The debriding head of the apparatus preferably has an oval shaped, egg-shaped or triangular shaped head with rounded corners that can be easily inserted and withdrawn from the mouth of the patient. The head is also hollow with an interior that is open to a tubular connection section that is configured to connect to a vacuum source and collection vessel. The shaped head also has at least one flattened lower surface with one or more intake openings that allow debris, fluids and airflow to pass from the openings through the head and tubular connection section to the collection vessel for disposal.

The flattened surface of the head also has one or more rows of bristles, blunt barbs or flexible spines with rounded tips that are associated in proximity to the openings of the head. The rows of bristles are preferably in parallel formation with the elongate openings in the head. In other embodiments, the openings are circular or oval and oriented linearly with rows of bristles. In other embodiments the bristles and openings are oriented randomly on the flattened surface of the head. The bristles may be made of materials that are flexible or semi-rigid.

The debris removal portion of the system has a vacuum source that is coupled to a collection vessel and debriding head with collection tubes. The collection vessel is preferably a sealed container with and input duct and a vacuum duct that extends into the interior of the collection vessel. In one preferred embodiment, the input duct and the vacuum duct have pressure sensitive ball valves that are configured to close with abrupt changes in internal pressure within the vessel. These valves effectively stop the backflow of collected materials from the vessel to the head or out of the vessel to the vacuum source in this embodiment.

The apparatus head and collection structures for debriding of tongue debris and micro biota may be disposable or may be capable of disinfection and reuse. The debriding head is preferably made from a semi-rigid or rigid material that will not significantly deform in the presence of the internal vacuum pressure or bend excessively when the head is applied to the tongue during use.

In one embodiment the debriding tool is configured to attach to dental high-volume evacuators (HVE) which are used by dentists during dental procedures to remove debris and various fluids from the oral cavity. In this embodiment the tool attaches to the active end of the HVE tube thereby converting it to an effective tongue cleaner.

In one embodiment the tool includes stiff rounded bristles that allow for gentle scrubbing of the dorsal surface of the tongue and thereby dislodge dental debris and biota that have established in the crevices between the papillae. The HVE simultaneously suctions away the loose particles and fluids.

According to one aspect of the technology, a debriding head and system are provided that will effectively and safely, mechanically debride the dorsum of the tongue and remove debris and microorganisms from the oral cavity.

A further aspect of the technology is to provide a tongue debriding head that is adaptable to existing vacuum systems and simple to use.

Another aspect of the technology is to provide an apparatus that is inexpensive to manufacture, durable and disposable or reusable.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a top perspective view of the tongue debriding apparatus according to one embodiment of the technology.

FIG. 2 is a bottom perspective view of the tongue debriding apparatus of FIG. 1.

FIG. 3 is a top view of the tongue debriding apparatus of FIG. 1.

FIG. 4 is a side view of the tongue debriding apparatus of FIG. 1.

FIG. 5 is a bottom view of the tongue debriding apparatus of FIG. 1.

FIG. 6 is a front view of the tongue debriding apparatus of FIG. 1.

FIG. 7 is a top perspective view of a sealed collection vessel according to one embodiment of the technology.

FIG. 8 is a cross-sectional side view of the sealed collection vessel embodiment of FIG. 7.

FIG. 9 is a detailed cross-sectional view of a basket valve.

FIG. 10 is a top view of one embodiment of a flexible rubber seal component of one lid of the sealed collection vessel showing inlet and outlet ports.

FIG. 11 is a cross-sectional side view of an alternative embodiment of a sealed collection vessel according to the technology.

FIG. 12 is a schematic system view of a self-contained tongue debriding system according to one embodiment of the technology.

FIG. 13 is a perspective view of a self-contained, portable or tabletop embodiment for home care, veterinary care, nursing facility, research or similar applications.

FIG. 14 is a top view of a tongue debriding apparatus with a coupler on the end of the tubular section to allow the reversable or permanent coupling of the apparatus with a vacuum tube and vacuum source.

FIG. 15 is a side view of a tongue debriding apparatus with dimensions according to one embodiment of the technology.

FIG. 16 is a bottom view of the tongue debriding apparatus of FIG. 15 with dimensions.

FIG. 17 is a front view of the tongue debriding apparatus of FIG. 15 according to the invention.

### DETAILED DESCRIPTION

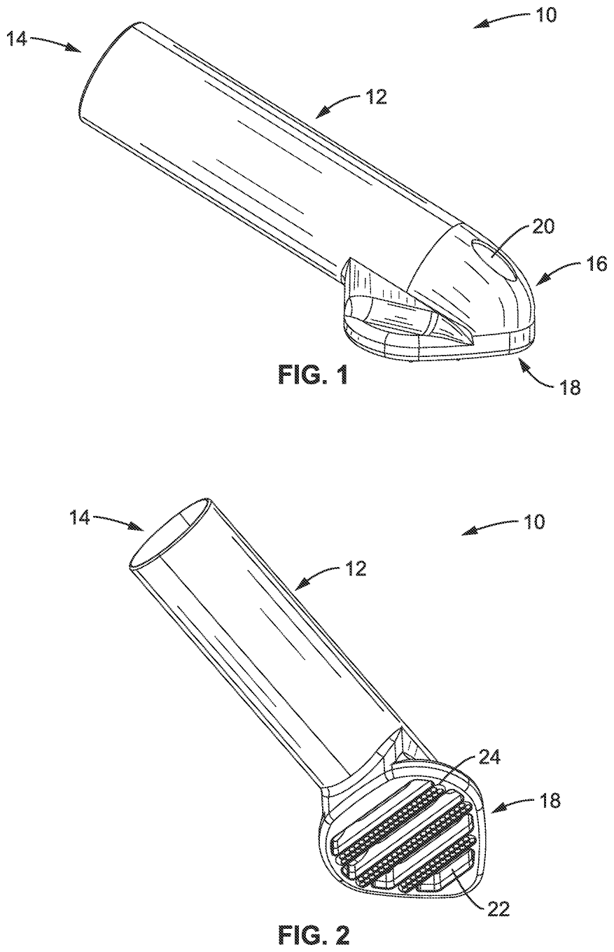

Referring more specifically to the drawings, for illustrative purposes, systems and methods for efficient and effective tongue debridement are generally shown. Several embodiments of the technology are described generally in FIG. 1 to FIG. 17 to illustrate the characteristics and functionality of the devices, systems and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

Turning now to FIG. 1, an embodiment of the apparatus 10 of the system for tongue and oral cavity debridement is shown schematically. The apparatus 10 has a generally cylindrical tubular body 12 with an open end 14 at one end of the body 12 and a debriding head 16 that extends from the other end of the tubular body 14. The debriding head 16 has a flattened lower surface 18 and an upper surface with a pressure release valve 20 in this embodiment.

Referring also to FIG. 2, the flat surface 18 of the debriding head 16 in this embodiment has four slotted openings 22 to the interior of the hollow debriding head 16 and tubular body 12. Debriding elements, in the form of elongate brushes 24, are positioned in between each of the slotted openings 22. The brushes 24 in the embodiment shown are formed from two parallel rows of bristles. While elongate brushes 24 are shown in this illustration, it will be understood that the debriding elements may take other forms such as groupings of bristles, pads, nubs, flexible spines or rubber scrapers and the like.

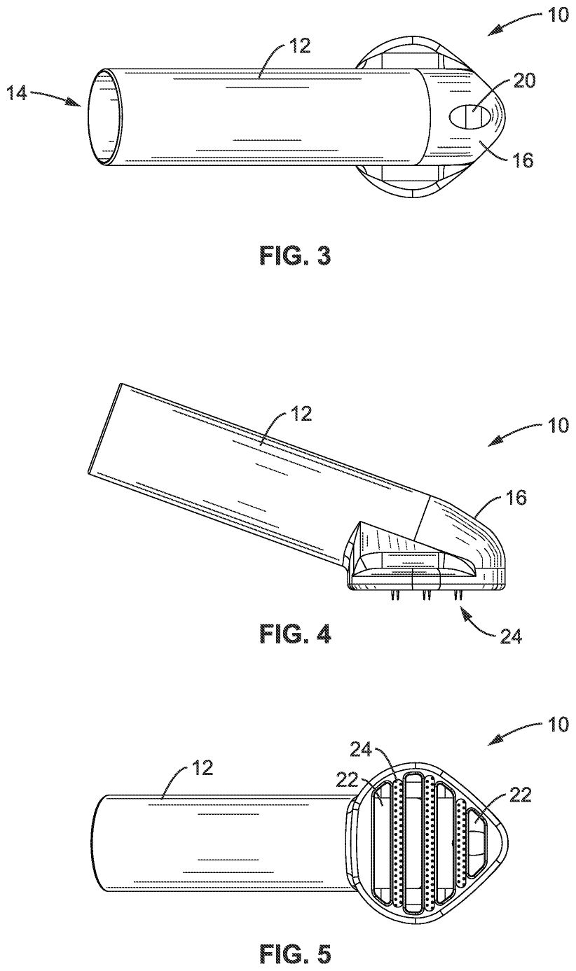

As seen in FIG. 3, the open end 14 of the tubular body 12 is configured to attach to a tube and vacuum source. The debriding elements of the head 16 dislodge debris from the tongue or other surface that is removed with negative pressure through the openings 22 of the head 16 from the vacuum source. Rubbing the debriding elements of surface 18 of the head 16 on the irregular surface of the tongue may close off the slotted openings 22 increasing the vacuum pressure in the interior of head 16. An increase in pressure on the tongue surface drawn into the slotted openings 22 during use may cause discomfort. The vacuum pressure release valve 20 of head 16 modulates the interior vacuum pressure avoiding overpressures and discomfort while maintain air flow through the system. The pressure release valve may be a pressure sensitive one way valve or simply a small opening in the head in some embodiments.

As seen in FIG. 4, the central axis of the tubular body 12 section is preferably oriented at an angle to the flat surface 18 of the debriding head 16. This angle aids in positioning the bristles of debriding element 24 of the flat section 18 of the head 16 on the surface of the tongue during use. The bristles of the debriding head 16 can be applied to the tongue surface with a back and forth or side to side motion or in a circular pattern.

The shape of the flat surface 18 and the debriding head 16 can vary. A rounded tear drop shape is illustrated in FIG. 5. The rounded edges of the head 16 reduce any discomfort from impacts to parts of the mouth during use. Although four slotted openings 22 and three debriding elements 24 are illustrated in this embodiment, fewer or more than four openings may be used. In another embodiment, the openings may be rows of circular holes or different numbers or sizes of holes. Other opening configurations are also possible.

The tubular body 12 and debriding head 16 are sized to fit easily in the mouth without eliciting the gag reflex. The opening 14 of the tubular body 12 is also sized to connect to a hose with a coupling. In one embodiment, the opening 14 of the tubular body 12 is sized to permit the insertion of the end of a conventional vacuum evacuator to frictionally attach the apparatus 10 to the evacuator.

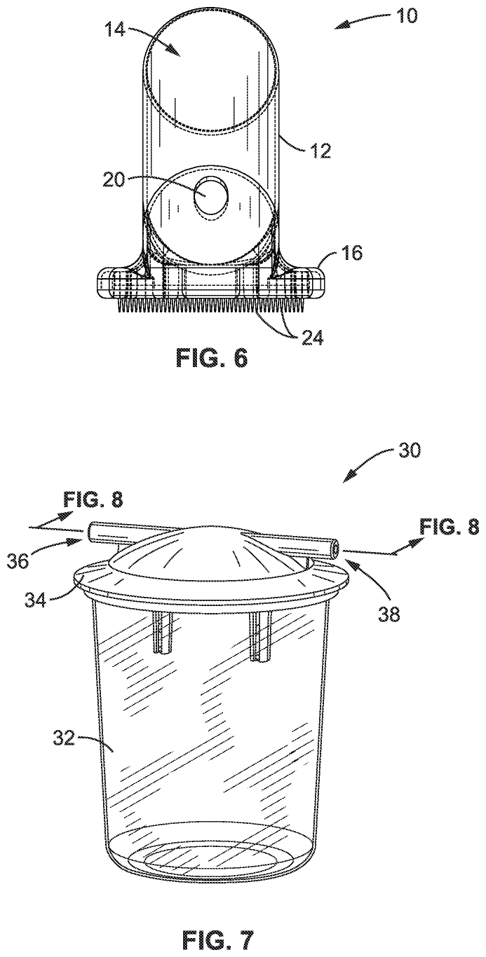

In another embodiment, the tubular body 12 is attached to a coupling and hose that are attached to an isolated vacuum source that is part of the system. The opening 14 of the tubular body 12 is preferably circular as shown in FIG. 6. However, the opening 14 can be a variety of shapes to accommodate couplings or shaped vacuum hose ends. The apparatus 10 is a hollow plastic instrument which may be produced in clear or in various different colors in one embodiment.

Turning now to FIG. 7 through FIG. 10, an embodiment of an in-line collection vessel 30 is shown schematically. The collection vessel 30 has a base container 32 and a lid 34 that can form an air-tight seal with the base container 32. In the embodiment shown in FIG. 7, the lid 34 has inlet connector 36 and an outlet connector 38. The inlet connector 36 and outlet connector 38 are sized and configured to be connected to hoses from the debriding apparatus 10 at the inlet connector 36 and the vacuum source at the outlet connector 38.

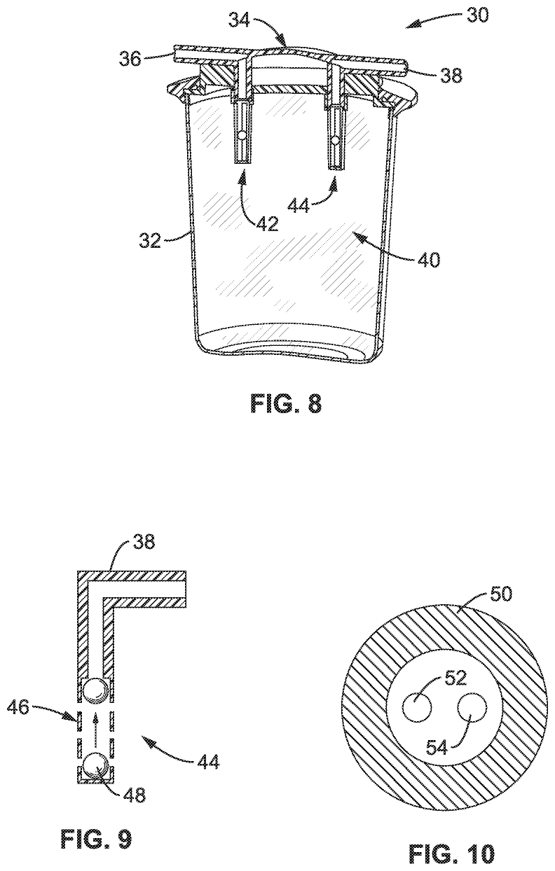

As seen in the cross-section of FIG. 8, the interior ends of the inlet connector 36 and the outlet connector 38 turn downward into the interior space 40 of the base container 32 of the collection vessel 30. At the interior end of the inlet connector 36 is an inlet basket valve 42. Likewise, at the interior end of the outlet connector 38 is an outlet basket valve 44.

As also shown in detail in FIG. 9, the basket valve 44 has a basket 46 enclosing a ball 46. In one embodiment, each cylindrical basket 46 houses a hollow light weight plastic ball 48. Changes in debris fluid levels within the interior 40 of the base container 32 or spiking pressure changes will cause the movement of the ball 48 withing the basket 46 and seat as shown by the arrow in FIG. 9. Seating of the ball 48 in the basket valve 44 will close off the connection between the interior 40 of the and the inlet 36 and outlet 38 connectors so that there will be no further flow of fluid through the system. Although the length of the inlet and outlet basket valves 42, 44 are shown in FIG. 8 as having equal lengths, it will be understood that the lengths of the connections or valves may be different.

The lid 34 and the base container 32 preferably use a seal 50 to form a negative pressure vacuum tight fit as shown in FIG. 10. In this embodiment, the basket valves can be inserted through the openings 52, 54 in the seal 50 to the interior 40 of the base container 32.

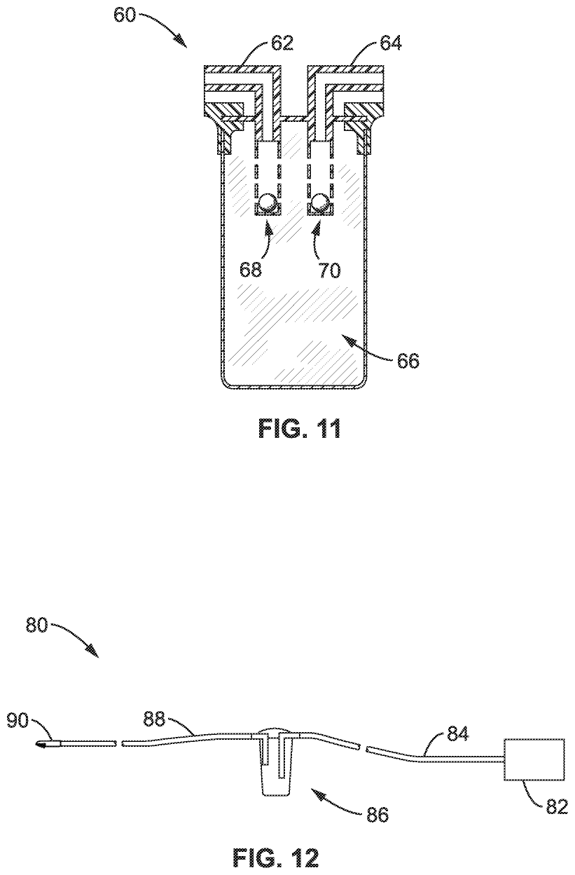

An alternative embodiment of a collection vessel 60 is shown in FIG. 11. In this embodiment, the lid and inlet-outlet connectors are a single integrated unit and the lid is not separable from the base container. The collection vessel 60 has an inlet connector 62 and an outlet connector 64. In one embodiment, the direction of flow through may be indicated by embossed directional arrows on the connectors. Once filled, the collection vessel 60 is disconnected from the input and vacuum lines and a new collection vessel is provided and inlet and outlet connectors are coupled to the to the corresponding lines.

Each connector 62, 64 turns downward 90° and extends down into the interior 66 of the vessel 60 in the embodiment of FIG. 11. The inlet connector 62 is attached internally to a firm resin cylindrical basket valve 68 and the outlet connector 64 is attached to basket valve 70 within the interior 66 of the vessel 60. Each cylindrical basket valve 68, 70 houses a hollow light weight plastic ball. Each ball rises in the basket as debris and oral fluids from tongue cleaning procedure rises in the collection vessel beyond the lower extremity of the basket valves 68, 70. When each ball rises to the connection point the upper aspect of the ball closes the opening which prevents fluid flowing out the outlet connector 64 and damaging the suction motor. This also simultaneously prevents backflow of fluid to the user via the inlet connector 62.

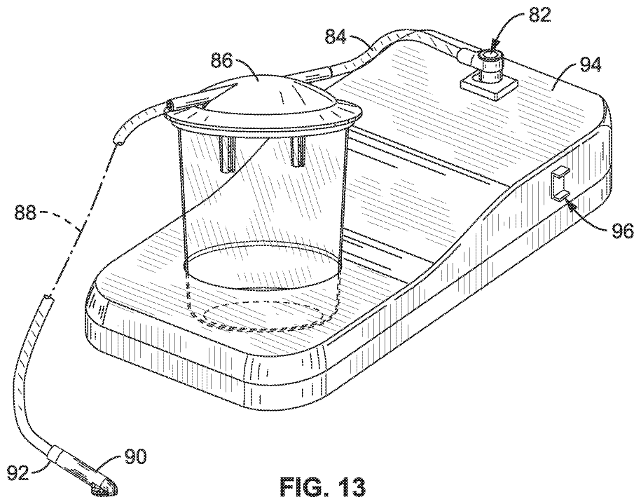

Referring now to FIG. 12 and FIG. 13, one embodiment of a self-contained system 80 is shown schematically. The system is illustrated with an embodiment adapted for use in a professional dental or veterinary setting for clarity. However, the components and system 80 can be miniaturized and adapted to a portable, self-contained handheld apparatus for home use. These compact systems, like that shown in FIG. 13, can be configured for home use, nursing facility use, veterinary office use or for research applications.

The system 80 has a vacuum source 82, such as a commercial compact suction motor, which provides the negative pressure (suction) necessary for the unit to work. The vacuum source 82 is connected to an air-tight collection jar 86 with a hose 84 that is preferably made of flexible rubber or silicon. In an alternative embodiment, the hose 84 is rigid and made from plastic or metal.

The collection jar 86 has a fluid flow prevention valve to prevent fluid flow and debris from flowing through the collection jar outlet through tube 84 into the suction pump and motor 82.

The inlet of the collection jar 86 is connected to the tongue debriding device 90 with an inlet hose 88. A backflow prevention valve is attached to the inlet of the collection jar 86 to prevent any backflow of material from the jar 86 to hose 88 and out through the device 90 with the release of negative pressure in the jar.

The system can also be consolidated and compacted into a portable or tabletop apparatus with the components in a housing. As illustrated in FIG. 13, the vacuum pump 82, hose 84, collection vessel 86, input hose 88 and the instrument 90 coupled to the end of the input hose with a coupler 92. The vacuum pump 82 of the apparatus can be actuated with an on-off switch 96. The self-contained unit illustrated in FIG. 13 can be portable or placed in a bathroom near a sink for home use or in a veterinary treatment setting, for example.

In use, the vacuum suction pump 82 of system 80 is activated with the switch 96 evacuating air from hose 84 and the interior of collection jar 86 creating a negative pressure in the collection jar 86. Concurrently, suction is created in the inlet hose 88 and debriding device 90 and the system is ready for use. The head of the activated debriding apparatus 90 is inserted into the mouth and the bristles and suction are applied to the tongue. In one embodiment, the insertion and use of the apparatus 90 is preceded with an application of water or antibacterial fluid to the tongue surfaces. The bristles of the debriding head of instrument 90 are then drawn across the tongue dislodging debris, microorganisms, fluids and coatings that are removed to the collection jar 86 through the openings of the debriding head and intake hose 88.

In another embodiment, water or other fluid may be applied simultaneously with the movements of the head from a separate source or from an optional sprayer coupled to the head of the apparatus 90. The presence of additional liquids during use supports the flow of debris, microorganisms and fluids from the treatment area to the collection jar 86 where it stored for disposal.

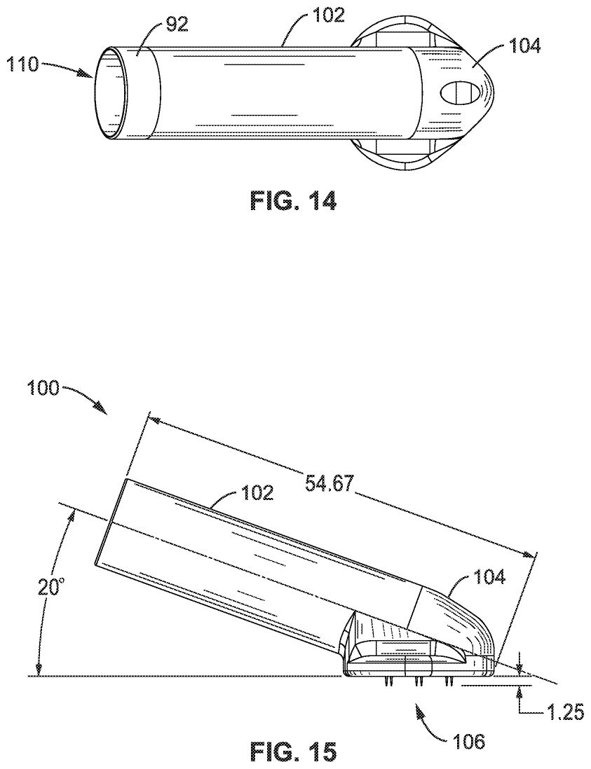

In another embodiment, the tongue debriding instrument 90 can be reversibly or permanently fixed to a vacuum tube with a coupler 92 as shown in detail in FIG. 14. The coupler 92 is positioned at the proximal end of the tubular section 102 to allow a vacuum hose (not shown) to be fixed to the end of the instrument. The coupler 92 can be a threaded coupling, a bayonet mount coupling, quick release coupling, compression coupling or similar coupling known in the art. In one embodiment, the coupling 92 includes an optional vacuum shut off valve that will start or stop the suction through the openings and interior of the debridement apparatus. The on-off valve allows control over the amount of suction created by the instrument from the suction pump.

The technology described herein may be better understood with reference to the accompanying examples, which are intended for purposes of illustration only and should not be construed as in any sense limiting the scope of the technology described herein as defined in the claims appended hereto.

### EXAMPLE

In order to demonstrate the functionality of the devices and procedures, a tongue debriding apparatus was fabricated and tested. Referring now to FIG. 14 through FIG. 17, the apparatus features are shown schematically and the numbered dimensions are shown in millimeters or degrees. The apparatus comprised two sections; a first section comprising a cylindrical tubular body, and a second section comprising a debriding head that extends from one end of the tubular body. The debriding head of the instrument had a flat surface with one or more openings and associated cleaning elements such as rubber, nylon or organic bristles, pads or scrapers.

As shown in the side view of FIG. 15, the length of the apparatus 100 can vary but, in the embodiment shown, the apparatus was about 55 mm in length. The length of the tubular body 102 section can vary but, in this embodiment, the tubular body 102 is about 47 mm in length.

The central axis of tubular body 102 was angled with respect to the horizontal portion 106 of the debriding head 104 with the central axis at approximately 20° from the horizontal in this illustration.

Rows of parallel bristles 106 extended beyond the surface of the flat section of the debriding head 104 as seen in FIG. 15 and FIG. 17. In the embodiment shown, the bristles have a length of 1.25 mm. Preferably the bristles or other debriding structures extend beyond the flat head surface in the range of between approximately 1 mm and approximately 2 mm.

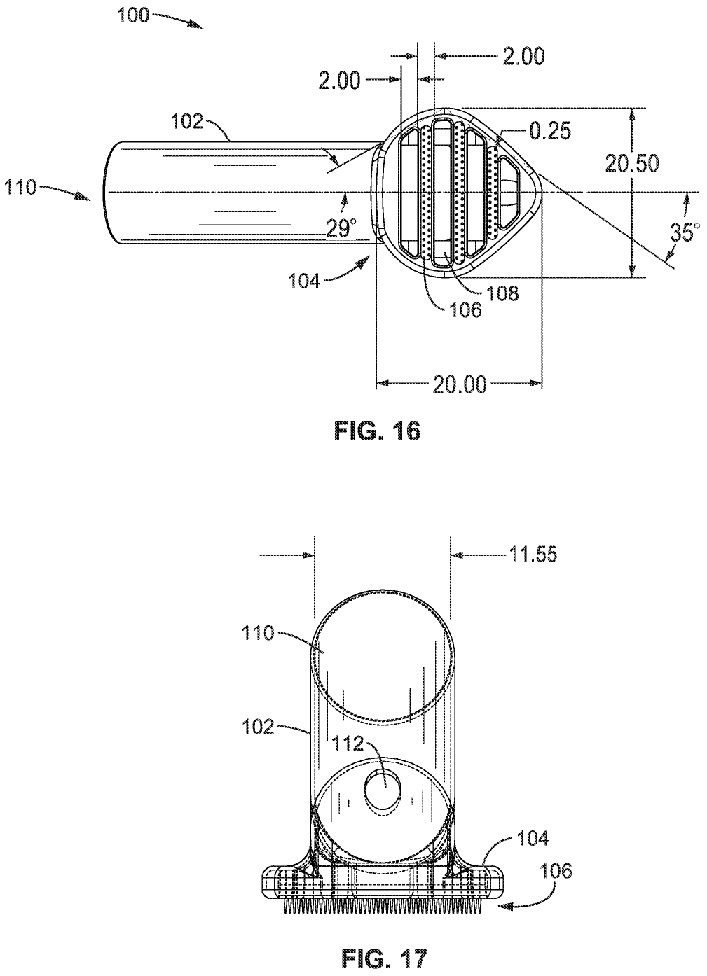

The shape of the debriding head 104 can vary but in one embodiment the debriding head has a rounded tear drop shape as illustrated in FIG. 16. The size of the debriding head 104 can vary but in the embodiment shown in FIG. 16 the debriding head 104 was about 20.5 mm at its widest point and about 20 mm in length with rounded points of the triangle shape. In this embodiment the tubular section curved about 160 degrees to the long axis and opened up seamlessly at one end into the debriding head 104.

In the embodiment shown in FIG. 16, the debriding head 104 comprises a flat lower active surface with four parallel trapezoidal-shaped openings 108 that were about 1.5 mm to about 2 mm wide, which amplified the suction force of the vacuum source (e.g. HVE). The lengths of these four openings 108 from front to back (distal to proximal) were about 7 mm, about 14 mm, about 16 mm and about 14 mm at their longest edge.

In the embodiment shown in FIG. 16, the two distal openings 108 at the front portion of the head 104 have lateral edges that are angled about 35 degrees to the long axis of the device as indicated. In this embodiment, the two proximal openings 108 have lateral walls that are angled about 29 degrees to the long axis of the device. In this embodiment, the trapezoidal openings 108 were spaced about 2 mm apart and do not extend to the edge of the device.

As seen in FIG. 16 and FIG. 17, there are three solid areas about 2 mm wide located between the parallel trapezoidal openings that have debriding elements mounted thereto. Here, in each of the areas there are two rows 106 of round-tipped nylon bristles of dimensions of about 1 mm to about 2 mm in length by about 0.25 mm in width. These stiff bristles 106 scrub the tongue surface, gently open crevices, and dislodge debris and biofilms. The stiffness of the bristles 106 may also reduce the tendency for gagging during use. Loose debris and biofilms were simultaneously suctioned through the slots and out into the central vacuum line of the HVE. Debris could be removed immediately without the patient seeing the evacuation in this configuration.

The other end of the tubular section 102 from the head 104 was a connection end 110 that was configured for attachment to a dental high-volume evacuator (HVE). As illustrated also in FIG. 17, the connecting end of the tubular section 102 may be smooth and cylindrical. While the diameter of the connecting end 110 can vary, in this illustration the diameter was about 11.5 mm. This size selection allowed a standard-sized vented HVE end to slide into the apparatus 100 with the vented side up.

The tubular section 102 may include a smooth upper internal surface that is interrupted by an about 1 mm to about 1.5 mm convexity positioned about 13 mm from orifices in the debriding head 104. This internal area engages the vent of the HVE as the device occludes the vent hole of the HVE. By engaging the vent hole of the HVE, displacement and possible aspiration of the device during function is prevented.

As also illustrated in FIG. 17, a device vent 112 is preferably positioned in the upper surface of the tubular section 102 about 46 mm from the connecting end 110 of the tubular body 102. In one embodiment the vent 112 was triangular shaped and about 5 mm from base to apex and about 5 mm wide at the base. In one embodiment, the apex was pointed toward the front of the base and the points of the triangular shaped vent, like the base, were also rounded. The vent 112 serves to prevent the device establishing a vacuum lock with the tongue during use and allows for a smooth scrubbing motion forward and backward.

Accordingly, in its various embodiments the apparatus is an inexpensive, easy-to-use, non-bulky, disposable dental tool for professional debriding of tongue debris and micro biota by dentists and dental clinical staff. The tool can be attached to dental high-volume evacuators (HVE) which are used during dental procedures to remove debris and various fluids from the oral cavity. In a preferred use the apparatus to the active end of the HVE thereby converting the HVE into an effective tongue cleaner.

The stiff rounded bristles 106 allowed gentle scrubbing of the dorsal surface of the tongue dislodging debris and biota that have established in the crevices between the papillae. The HVE simultaneously suctioned away the loose particles. Conversion of the HVE to use for tongue debriding was also fast and inexpensive for the professional dental or veterinary office/clinic.

From the description herein, it will be appreciated that the present disclosure encompasses multiple implementations of the technology which include, but are not limited to, the following:

A tongue debriding apparatus for use with a suction device, the apparatus comprising: a tubular section having a hollow interior; and a head section extending from the tubular section, the head section having a hollow interior; the head section having a debriding surface comprising a plurality of spaced apart openings separated by surface areas having bristles; the tubular section having a suction end configured for connection to a suction device; wherein the hollow interior of the tubular section is in fluidic communication with the hollow interior of the head section, and wherein suction applied to the suction end of the tubular section is drawn through the openings in the head section.

The apparatus of any preceding or following implementation, the head section further comprising a vent configured to prevent the head section from forming a suction lock with the tongue of a patient.

The apparatus of any preceding or following implementation, wherein the spaced apart openings in the head section have a shape configured to amplify suction force from the suction device.

The apparatus of any preceding or following implementation, wherein the bristles comprise parallel rows of bristles.

The apparatus of any preceding or following implementation, wherein the bristles are made of a material selected from the group of materials consisting of nylon, rubber and organic fibers.

The apparatus of any preceding or following implementation, the tubular section further comprising: a coupling positioned on the suction end of the tubular section opposite the head section, the coupling configured to couple the apparatus to a hose.

The apparatus of any preceding or following implementation, wherein the coupling is selected from the group of couplings consisting of a threaded coupling, a bayonet mount coupling and a compression coupling.

The apparatus of any preceding or following implementation, wherein the coupling further comprises an on-off valve.

A tongue debriding system, the system comprising: (a) a suction device; and (b) a tongue debriding instrument fluidly coupled to the suction device, the instrument comprising: (i) a tubular section having a hollow interior, the tubular section connected to the suction device; and (ii) a head section with a hollow interior extending from the tubular section, the head section having a flattened surface with a plurality of spaced apart openings to the hollow interior of the head section and a plurality of cleaning elements mounted between the spaced apart openings; (c) wherein suction applied to the suction end of the tubular section is drawn through the openings in the head section to the interior of the instrument.

The system of any preceding or following implementation, wherein the cleaning elements of the head section comprise one or more rows of bristles with a length extending outwardly from the head section.

The system of any preceding or following implementation, wherein the bristles are made of a material selected from the group of materials consisting of nylon, rubber and organic fibers.

The system of any preceding or following implementation, wherein the spaced apart openings in the head section of the instrument have a shape configured to amplify suction force from the suction device.

The system of any preceding or following implementation, wherein the head section of the instrument further comprises a vent configured to prevent the head section from forming a suction lock with the tongue of a patient.

A tongue debriding system, the system comprising: (a) a vacuum source; (b) a sealed collection vessel fluidly coupled to the vacuum source, the vacuum source configured to create a negative pressure in an interior of the collection vessel; and (c) a tongue debriding instrument fluidly coupled to the sealed collection vessel the tongue debriding instrument comprising: (i) a tubular section having a hollow interior, the tubular section connected to the sealed collection vessel; and (ii) a head section with a hollow interior extending from the tubular section, the head section having a flattened surface with a plurality of spaced apart openings to the hollow interior of the head section and a plurality of cleaning elements mounted between the spaced apart openings; (d) wherein negative pressure from the sealed collection vessel creates negative pressure in the interior of the head section creating suction through the openings to the interior of the head section; and (e) wherein particles or fluids drawn into the interior of the head section is collected in the sealed collection vessel.

The system of any preceding or following implementation, wherein the sealed collection vessel further comprises: an inlet valve fluidly connected to the tongue debriding instrument; and an outlet valve fluidly connected to the vacuum source; wherein movement of contents of the collection vessel out of the vessel by a change in pressure is prevented.

The system of any preceding or following implementation, wherein the inlet valve and the outlet valve are basket valves with buoyant balls.

The system of any preceding or following implementation, wherein the cleaning elements of the head section comprise one or more rows of bristles with a length extending outwardly from the head section.

The system of any preceding or following implementation, wherein the bristles are made of a material selected from the group of materials consisting of nylon, rubber and organic fibers.

The system of any preceding or following implementation, wherein the spaced apart openings in the head section of the instrument have a shape configured to amplify suction force from the suction device.

The system of any preceding or following implementation, wherein the head section of the instrument further comprises a vent configured to prevent the head section from forming a suction lock with the tongue of a patient.

As used herein, term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed.

Accordingly the reader should consult the application as filed for the original content of the disclosure. Any deletion of content of the disclosure should not be construed as a disclaimer, forfeiture or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. A tongue debriding apparatus for use with a suction device, the apparatus comprising:
a tubular section having a hollow interior; and
a head section extending from the tubular section, the head section having a hollow interior;
the head section having a debriding surface comprising a plurality of spaced apart openings separated by surface areas having bristles;
the tubular section having a suction end configured for connection to a suction device;
wherein the hollow interior of the tubular section is in fluidic communication with the hollow interior of the head section, and wherein suction applied to the suction end of the tubular section is drawn through the openings in the head section.

2. The apparatus of claim 1, the head section further comprising a vent configured to prevent the head section from forming a suction lock with the tongue of a patient.

3. The apparatus of claim 1, wherein the spaced apart openings in the head section have a shape configured to amplify suction force from the suction device.

4. The apparatus of claim 1, wherein said bristles comprise parallel rows of bristles.

5. The apparatus of claim 1, wherein said bristles are made of a material selected from the group of materials consisting of nylon, rubber and organic fibers.

6. The apparatus of claim 1, said tubular section further comprising:
a coupling positioned on the suction end of said tubular section opposite said head section, said coupling configured to couple said apparatus to a hose.

7. The apparatus of claim 6, wherein said coupling is selected from the group of couplings consisting of a threaded coupling, a bayonet mount coupling and a compression coupling.

8. The apparatus of claim 6, wherein said coupling further comprises an on-off valve.

9. A tongue debriding system, the system comprising:
(a) a suction device; and
(b) a tongue debriding instrument fluidly coupled to the suction device, the instrument comprising:
(i) a tubular section having a hollow interior, said tubular section connected to the suction device; and
(ii) a head section with a hollow interior extending from the tubular section, the head section having a flattened surface with a plurality of spaced apart openings to the hollow interior of the head section and a plurality of cleaning elements mounted between said spaced apart openings;
(c) wherein suction applied to the suction end of the tubular section is drawn through the openings in the head section to the interior of the instrument.

10. The system of claim 9, wherein the cleaning elements of the head section comprise one or more rows of bristles with a length extending outwardly from the head section.

11. The system of claim 10, wherein said bristles are made of a material selected from the group of materials consisting of nylon, rubber and organic fibers.

12. The system of claim 9, wherein the spaced apart openings in the head section of the instrument have a shape configured to amplify suction force from the suction device.

13. The system of claim 9, wherein the head section of the instrument further comprises a vent configured to prevent the head section from forming a suction lock with the tongue of a patient.

14. A tongue debriding system, the system comprising:
(a) a vacuum source;
(b) a sealed collection vessel fluidly coupled to the vacuum source, the vacuum source configured to create a negative pressure in an interior of the collection vessel; and
(c) a tongue debriding instrument fluidly coupled to the sealed collection vessel said tongue debriding instrument comprising:
(i) a tubular section having a hollow interior, said tubular section connected to the sealed collection vessel; and
(ii) a head section with a hollow interior extending from the tubular section, the head section having a flattened surface with a plurality of spaced apart openings to the hollow interior of the head section and a plurality of cleaning elements mounted between said spaced apart openings;
(d) wherein negative pressure from the sealed collection vessel creates negative pressure in the interior of said head section creating suction through the openings to the interior of the head section; and
(e) wherein particles or fluids drawn into the interior of the head section is collected in the sealed collection vessel.

15. The system of claim 14, wherein the sealed collection vessel further comprises:
an inlet valve fluidly connected to the tongue debriding instrument; and
an outlet valve fluidly connected to the vacuum source;
wherein movement of contents of said collection vessel out of said vessel by a change in pressure is prevented.

16. The system of claim 15, wherein the inlet valve and the outlet valve are basket valves with buoyant balls.

17. The system of claim 14, wherein the cleaning elements of the head section comprise one or more rows of bristles with a length extending outwardly from the head section.

18. The system of claim 17, wherein said bristles are made of a material selected from the group of materials consisting of nylon, rubber and organic fibers.

19. The system of claim 14, wherein the spaced apart openings in the head section of the instrument have a shape configured to amplify suction force from the suction device.

20. The system of claim 14, wherein the head section of the instrument further comprises a vent configured to prevent the head section from forming a suction lock with the tongue of a patient.
